(19) United States
(12) Patent Application Publication
(10) Pub. No.: US 2025/0195744 A1
(43) Pub. Date: Jun. 19, 2025

(54) SHIELDING CONTAINER FOR TRANSPORTING A RECEPTACLE CONTAINING A RADIOACTIVE PRODUCT

(71) Applicant: LEMER PAX, LA CHAPELLE SUR ERDRE (FR)

(72) Inventor: Pierre-Marie LEMER, Nantes (FR)

(21) Appl. No.: 18/847,900

(22) PCT Filed: Mar. 16, 2023
(86) PCT No.: PCT/EP2023/056812

§ 371 Date: Sep. 17, 2024

(30) Foreign Application Priority Data
Mar. 17, 2022 (FR) FR2202341

(51) Int. Cl. A61M 5/00; G21F 5/06

(52) U.S. Cl. CPC A61M 5/002; G21F 5/06; A61M 2209/06

(57) ABSTRACT

The invention relates to a shielding container (1) designed to transport a receptacle containing a radioactive product, the container comprising a container body (2), the container body being associated with a container lid (3) by means of a lid hinge axis (4). The container (1) comprises a handle structure (5) for gripping and carrying same, the handle structure being formed by a grip member (51) extended by two side arms (52) of the handle. The side arms (52) of the handle are rigidly attached to the end walls (24) of the container body (2) by means of a handle hinge axis (6); and said arms comprise a guide slot (522) which co-operates with lug structures (7) that are rigidly attached to the end sides of the lid (34) in order to ensure that the container lid (3) pivots about its hinge axis (4) by means of the lug structure (7) whenever the handle structure (5) pivots about the handle hinge axis (6).

### TECHNICAL FIELD OF THE INVENTION

The present invention relates to the technical field of equipment for protecting individuals against ionising radiation.

More particularly, it relates to a shielded container made of a radioprotective material, designed to transport a vessel containing a radioactive product, for example a syringe for injecting a radioactive product into a patient.

### STATE OF THE ART

In the above field, shielded containers are known, which are adapted to transport a syringe or a vial containing a radiopharmaceutical product to be injected into a patient.

These shielded containers, made at least partially of a radio-protective material, can be in the form of a receptacle, forming the container body, associated with a container lid.

The receptacle (or container body) comprises a bottom element bordered by a front wall, a rear wall and two end walls, which delimit together a packaging volume for the syringe and a container body opening.

The container lid may be rigidly attached to the container body via a hinge axis allowing it to move between a closed position, ensuring the desired radioprotection, and an open position, for the introduction or extraction of the vessel containing the radiopharmaceutical product.

However, such shielded containers are rather heavy (up to 15 kg) and the container lid is not easy to manoeuvre, with the consequent risk of finger being pinched or even injured.

### DISCLOSURE OF THE INVENTION

In order to remedy the above-mentioned drawbacks of the state of the art, the present invention proposes a shielded container, made at least partially of a radioprotective material for protection against ionising radiations, for transporting a vessel containing a radioactive product, for example a syringe for injecting a radioactive product into a patient, said container comprising:
a container body comprising a container body bottom element, bordered by a container body front wall, a container body rear wall and two container body end walls, delimiting together a packaging volume and a container body opening,
a container lid comprising a lid top element bordered by a lid front side, a lid rear side and two lid end sides, said container lid being rigidly attached to said container body via a lid hinge axis allowing it to move with respect to said container body between:
a closed position in which said container body closes said container body opening, and
an open position in which said container lid clears said container body opening to allow access to said packaging volume,
said shielded container being characterized in that it comprises a handle structure designed to be gripped for carrying it, said handle structure comprising a longitudinal grip member, extended by two side handle arms rigidly attached to the container body end walls via a handle hinge axis,
said container also comprising manoeuvring means for manoeuvring said container lid between said open and closed positions,
said manoeuvring means comprising at least one lug structure rigidly attached to one lid end side, said lug structure being able to move in a guiding slot formed in the side arm opposite said handle structure,
said guiding slot being so shaped to pivotally manoeuvre said container lid about said lid hinge axis, through said lug structure, by pivotally manoeuvring said handle structure about said handle hinge axis.

Thus, the container lid is manoeuvred in a controlled and safe manner by pivoting the handle structure.

Other non-limiting and advantageous features of the shielded container according to the invention, taken individually or according to all the technically possible combinations, are the following:
the guiding slot has the shape of an arc of a circle whose centre is located on the side of the lid hinge axis;
one of the ends of the guiding slot forms a stop means for the associated lug structure, in a suitable way to prevent the container lid from opening beyond 90°;
the two side handle arms comprise a guiding slot each cooperating with a lug structure rigidly attached to the corresponding lid end side of the container lid;
the lug structure(s) are formed on the associated lid end side, between the lid hinge axis and the lid front side;
the handle hinge axis is arranged near the container body front wall;
the container body comprises a longitudinal median plane that extends parallel to said container body front and rear walls and that passes through said container body end walls in their central area,
and said longitudinal grip member of the handle structure extends, in closed position of the container lid, in said longitudinal median plane or approximately in said longitudinal median plane;
the container body bottom element, the container body front wall, the container body rear wall, the container body end walls and the lid top element comprise a lead core covered by inner and outer linings made of a material that is less radioprotective than lead, such as stainless steel for example;
the shielded container comprises a plate added against the outer face of the container body bottom element, said plate comprising an anti-tilt extension that projects from the plane of the container body rear wall and that is arranged to prevent said shielded container from tipping over at the end of the container lid opening stroke;
the shielded container comprises a removable, liquid-tight packaging support arranged in the packaging volume, in rest on the container body bottom element, said packaging support being adapted to receive and wedge a vessel containing a radioactive product.

Of course, the different features, alternatives and embodiments of the invention can be associated with each other according to various combinations, insofar as they are not mutually incompatible or exclusive.

### DETAILED DESCRIPTION OF THE INVENTION

Moreover, various other features of the invention emerge from the appended description made with reference to the drawings that illustrate non-limiting embodiments of the invention, and wherein:

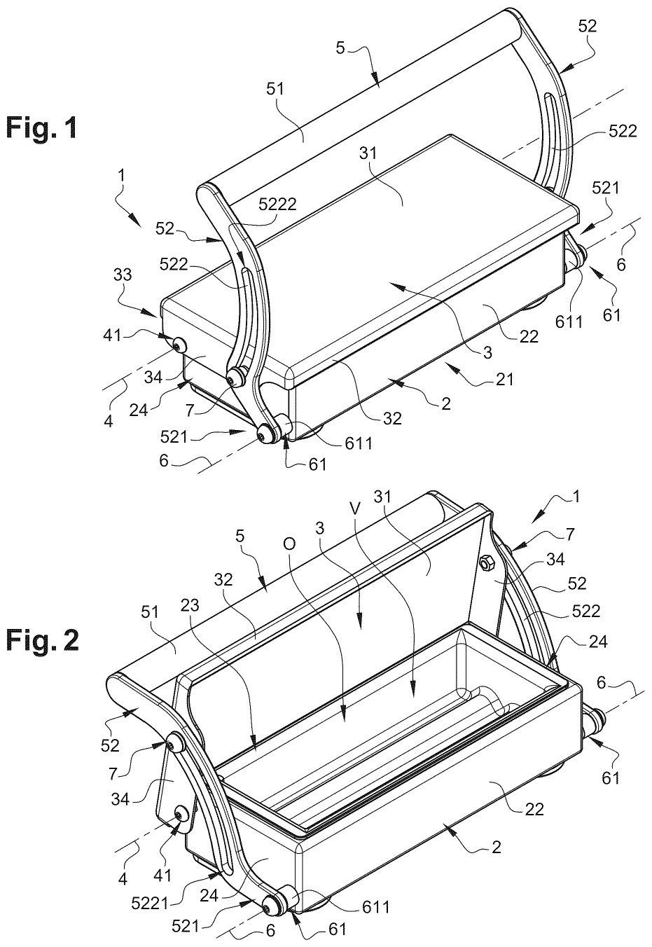

FIG. 1 is a perspective view of a possible embodiment of a shielded container in accordance with the invention, here shown with its container lid in closed position;

FIG. 2 is a perspective view of the shielded container illustrated in FIG. 1, here shown with its container lid in open position;

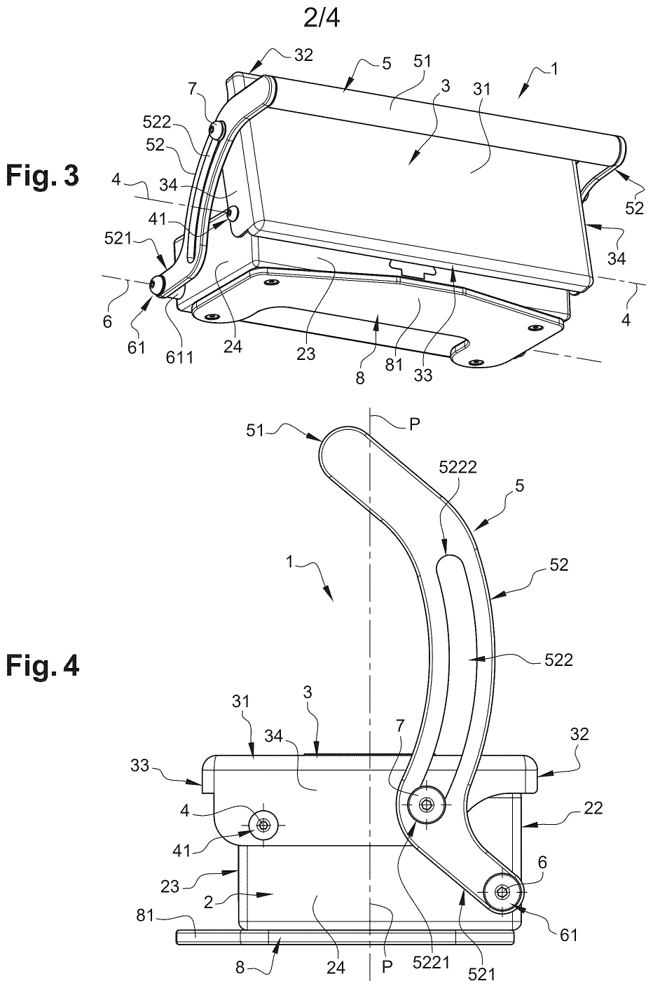

FIG. 3 shows the shielded container illustrated in FIG. 2, seen in perspective from another angle;

FIG. 4 is a side view of the shielded container illustrated in FIGS. 1 to 3, here shown with its container lid in closed position;

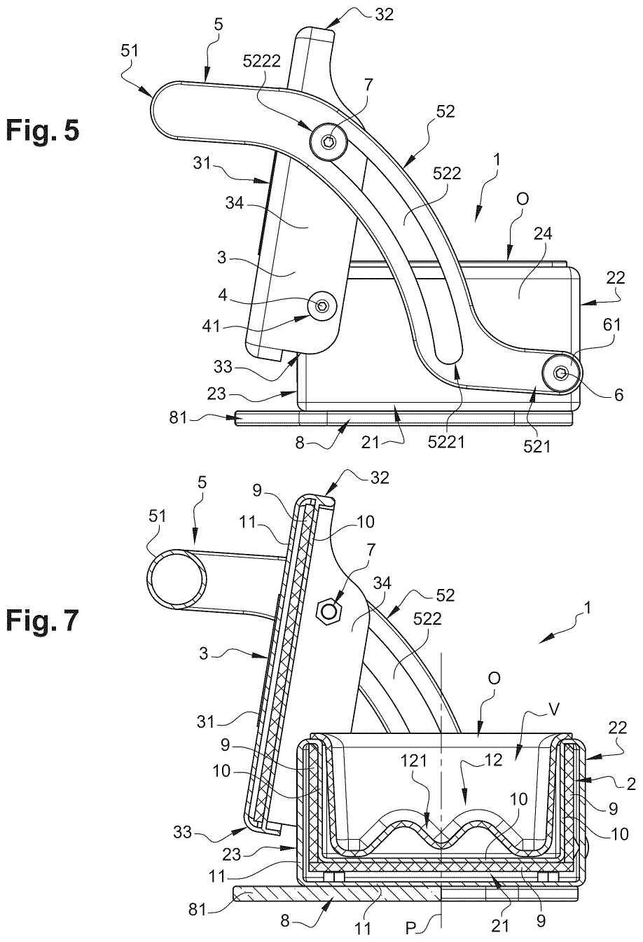

FIG. 5 is a side view of the shielded container illustrated in FIGS. 1 to 4, here shown with its container lid in open position;

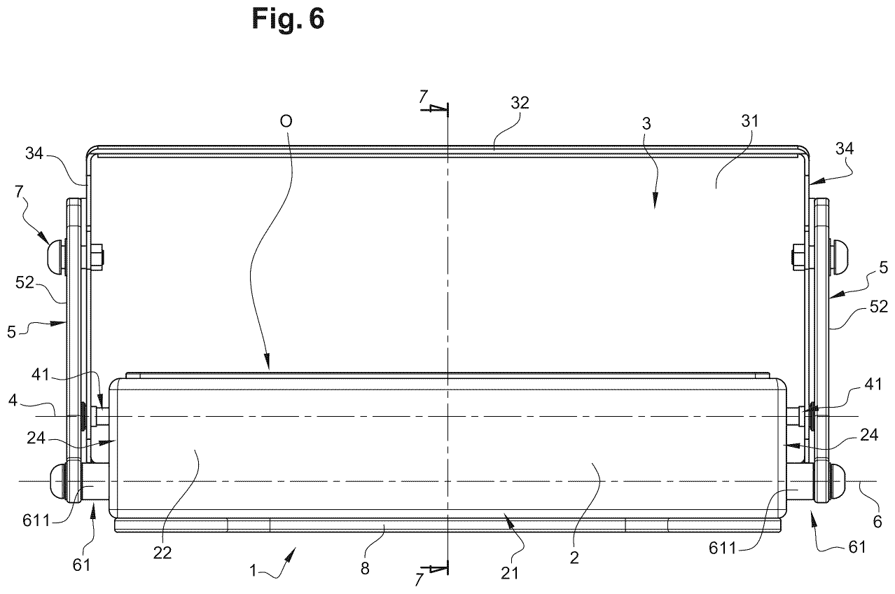

FIG. 6 is a front view of the shielded container illustrated in FIGS. 1 to 5, here shown with its container lid in open position;

FIG. 7 is a sectional view along the section plane 7-7 of the shielded container illustrated in FIG. 6.

The shielded container 1 illustrated in FIGS. 1 to 7 has the general shape of a suitcase adapted to transport a vessel (not shown) containing a radioactive product, for example a syringe or a vial containing a radiopharmaceutical product to be injected into a patient.

This shielded container 1 comprises:
a container body 2,
a container lid 3, hinged to the container body 2 about a lid hinge axis 4, and
a handle structure 5, which can be gripped for carrying the shielded container 1, and which also allows manoeuvring the container lid 3 between its open and closed positions with respect to the container body 2, as explained hereinafter in the description.

The container body 2 comprises a flat container body bottom element 21, bordered by a container body front wall 22, a container body rear wall 23 and two container body end walls 24.

The container body front wall 22, the container body rear wall 23 and the two container body end walls 24 all have the same height. They extend on the same side with respect to the plane of the container body bottom end 21 and they form together a belt of peripheral walls.

The container body bottom element 21 has a rectangular general shape; the container body front wall 22 and the container body rear wall 23 are parallel to each other; and the two container body end walls 24 are also parallel to each other.

The container body 2 comprises a longitudinal median plane P that extends parallel to the container body front and rear walls 22, 23 and that passes through the container body end walls 24 in their central area.

The various constituent parts 21, 22, 22 and 24 of the container body 2 delimit together a packaging volume V and a container body opening O.

For its part, the container lid 3 comprises a flat lid top element 31, bordered by a lid front side 32, a lid rear side 33 and two lid end sides 34.

The lid front side 32, the lid rear side 33 and the two lid end sides 34 form together a peripheral edge that extends on one side of the plane of the lid top element 31.

The lid top element 31 has a rectangular general shape; the lid front side 32 and the lid rear side 33 are parallel to each other; and the two lid end sides 34 are also parallel to each other.

The container lid 3 is rigidly attached to the container body 2 via the lid hinge axis 4, which allows it to move between:
a closed position (visible in FIGS. 1 and 4) in which the container lid 3 closes the container body opening O, and
an open position (visible in FIGS. 2, 3, 5, 6 and 7) in which the container lid 3 clears the container body opening O to allow access to the packaging volume V.

In closed position, the lid top element 31 of the container lid 3 comes into rest on the upper edge of the belt of peripheral walls 22, 23, 24 of the container body 2; and the peripheral edge formed by the lid front side 32, the lid rear side 33 and the two lid end sides 34 surrounds this belt of peripheral walls 22, 23, 24.

The lid hinge axis 4 is arranged near the container body rear wall 23 and on the lid rear side 33. It is materialized by two lid pivot structures 41 connecting, on either side of the container 1, a lid end side 34 and a container body end wall 24.

Each lid pivot structure 41 may consist of a screw-nut type connection system that passes through the lid end side 34 and the container body end wall 24.

The handle structure 5 of the container 1 comprises a longitudinal grip member 51, extended by two side handle arms 52, rigidly attached to the container body end walls 24.

As mentioned hereinabove, the handle structure 5 is not only adapted to allow gripping the container 1, but also to ensure the manoeuvre of the container lid 3 between its open and closed positions.

For that purpose:
the ends 521 of the two side handle arms 52 are rigidly attached to the container body end walls 24 via a handle hinge axis 6, and
the means for manoeuvring the container lid 3 comprise, on each side of the container 1, a lug structure 7 rigidly attached to one lid end side 34, which is able to move in a guiding slot 522 formed in the side arm 52 opposite the handle structure 5.

The lug structure 7/guiding slot 522 couples are identical on each side of the container 1.

The positioning of the lug structures 7 on the lid end sides 34, and the shape of the guiding slots 522, are adapted to ensure the pivoting manoeuvre of the container lid 3 about the lid hinge axis 4, via the lug structures 7, by pivotally manoeuvring the handle structure 5 about the handle hinge axis 6.

In this context, the lug structures 7 are formed on the associated lid end side 34, between the lid hinge axis 4 and the lid front side 32.

On the other hand, each guiding slot 522 has the shape of an arc of a circle whose centre is located on the side of the lid hinge axis 4.

The handle hinge axis 6 is materialized by two handle pivot structures 61 connecting, on each side of the container 1, the end 521 of the side handle arm 52 and the opposite container body end wall 24.

Each handle pivot structure 61 may consist of a screw-nut type connection system that passes through the side handle arm 52 (at the end 521 thereof) and the opposite container body end wall 24. A spacing washer 611 is provided between the end 521 of the side handle arms 52 and the opposite container body end wall 24, to take into account the length difference between the container lid 3 and the container body 2.

As can be seen in the appended figures, the handle hinge axis 6 is arranged near the container body front wall 22.

Each lug structure 7 may consist of a screw-nut type system that passes through the opposite lid end side 34. The nut of this screw-nut type system is here positioned inside the container lid 3; the screw body forms the element that circulates in the associated guiding slot 522; and the screw head forms a stop member that prevents the side handle arm 52 and the container lid 3 from detaching from each other.

For reasons of movement stability and fluidity, in particular, the two sides of the container 1 are preferably equipped with a lug structure 7/guiding slot 522 couple, as illustrated in the figures; however, as the case may be, in an alternative embodiment, only one of the two container sides 1 may be equipped with such a lug structure 7/guiding slot 522 couple.

The two ends of the guiding slots 522 serve as a stop means for the lug structures 7 and define the open and closed stable positions of the container lid 3.

More precisely, the end 5221 of the guiding slot 522, which is located on the side of the handle hinge axis 6, serves as a stop means for the lug structures 7 in order to define the stable closed position of the container lid 3 (FIGS. 1 and 4).

To facilitate and optimize the operations of hand-carrying the container 1, in this stable closed position, the longitudinal grip member 51 of the handle structure 5 extends in the longitudinal median plane P (or approximately in this longitudinal median plane P) of the container body 2.

For its part, the end 5222 of the guiding slot 522, which is located remote from the handle hinge axis 6, serves as a stop means for the lug structures 7 in order to define the stable open position of the container lid 3 (FIGS. 2, 3, 5, 6 and 7).

It is observed that the end 5222 of the guiding slot 522 prevents the opening of the container lid 3 beyond 90° in order to ensure the stability of the container 3 and limit the risk of it tipping over when placed on a support.

More precisely, this end 5222 of the guiding slot 522 limits the opening of the container lid 3 to an angular sector slightly lower than 90° (of the order of 85° to) 88°.

Still with the aim of optimising stability, the shielded container 1 comprises a plate 8 added against the outer face of the container body bottom element 21; and this plate 8 comprises an anti-tilt extension 81 that projects from the plane of the container body rear wall 23.

This plate 8 is attached by any suitable means against the outer (or underside) face of the container body bottom element 21, for example by screwing.

It is understood that the anti-tilt extension 81, projecting from the side where the container lid 3 is hinged to the container body 2, avoids or at least limits the risk for the shielded container 1 to tip over at the end of the container lid 3 opening stroke.

The container 1 is made at least partially of a radioprotective material, in such a way that, when it is in closed position, it protects the environment, and in particular the individuals in proximity, against the ionising radiations emitted by the packed radioactive product.

As illustrated in the sectional view of FIG. 7, the container body bottom element 21, the container body front wall 22, the container body rear wall 23, the container body end walls 24 and the lid top element 31 may comprise a lead core 9 covered by inner 10 and outer 11 linings made of a mechanically resistant material that is less radioprotective than lead (for example, in the form of covering plates of stainless steel).

Here, the peripheral edge of the container lid 3 formed by the lid front side 32, the lid rear side 33 and the two lid end sides 34 are made only of stainless steel.

In an alternative embodiment, the shielded container 1 may be made of solid tungsten.

Still in the sectional view of FIG. 7, it is noted that the shielded container 1 can comprise a packaging support 12 arranged in the packaging volume V, adapted to receive and wedge a vessel (for example, in the form of a syringe or a vial) containing the radioactive product.

This packaging support 12 is arranged in rest on the container body bottom element 21; and is designed to be removable and liquid-tight.

The packaging support 12 may be in the form of a containment tray adapted to collect any leaks of radioactive product, the bottom part 121 of which is adapted to receive and wedge the container containing the radioactive product.

In the embodiment illustrated, the bottom part 121 of the packaging support 12 is in the form of a cradle adapted to receive a syringe-type vessel.

1. A shielded container (1), made at least partially of a radioprotective material for protection against ionising radiations, for transporting a vessel containing a radioactive product, for example a syringe for injecting a radioactive product into a patient, said container (1) comprising:
a container body (2) comprising a container body bottom element (21), bordered by a container body front wall (22), a container body rear wall (23) and two container body end walls (24), delimiting together a packaging volume (V) and a container body opening (O),
a container lid (3) comprising a lid top element (31) bordered by a lid front side (32), a lid rear side (33) and two lid end sides (34),
said container lid (3) being rigidly attached to said container body (2) via a lid hinge axis (4) allowing it to move with respect to said container body (2) between:
a closed position in which said container body (3) closes said container body opening (O), and
an open position in which said container lid (3) clears said container body opening (O) to allow access to said packaging volume (V),
wherein said shielded container (1) comprises a handle structure (5) designed to be gripped for carrying it, said handle structure (5) comprising a longitudinal grip member (51), extended by two side handle arms (52) rigidly attached to the container body end walls (24) via a handle hinge axis (6),
said container (1) also comprising manoeuvring means (7, 522) for manoeuvring said container lid (3) between said open and closed positions,
said manoeuvring means comprising at least one lug structure (7) rigidly attached to one lid end side (34), said lug structure (7) being able to move in a guiding slot (522) formed in the side arm (52) opposite said handle structure (5),
said guiding slot (522) being so shaped to pivotally manoeuvre said container lid (3) about said lid hinge axis (4), through said lug structure (7), by pivotally manoeuvring said handle structure (5) about said handle hinge axis (6).

2. The shielded container (1) according to claim 1, wherein said guiding slot (522) has the shape of an arc of a circle whose centre is located on the side of said lid hinge axis (4).

3. The shielded container (1) according to claim 1, wherein one of the ends (5222) of said guiding slot (522) forms a stop means for the associated lug structure (7), in a suitable way to prevent said container lid (3) from opening beyond 90°.

4. The shielded container (1) according to claim 1, wherein the two side handle arms (52) comprise a guiding slot (522) each cooperating with a lug structure (7) rigidly attached to the corresponding lid end side (34) of said container lid (3).

5. The shielded container (1) according to claim 1, wherein said lug structure(s) (7) are arranged on the associated lid end side (34), between said lid hinge axis (4) and said lid front side (32).

6. The shielded container (1) according to claim 1, wherein said handle hinge axis (6) is arranged near said container body front wall (22).

7. The shielded container (1) according to claim 1, wherein said container body (2) comprises a longitudinal median plane (P) that extends parallel to said container body front and rear walls (22, 23) and that passes through the container body end walls (24) in their central area,
and wherein said longitudinal grip member (51) of said handle structure (5) extends, in closed position of the container lid (3), in said longitudinal median plane (P) or approximately in said longitudinal median plane (P).

8. The shielded container (1) according to claim 1, wherein said container body bottom element (21), said container body front wall (22), said container body rear wall (23), said container body end walls (24) and said lid top element (31) comprise a lead core (9) covered by inner (10) and outer (11) linings made of a material that is less radioprotective than lead, such as stainless steel for example.

9. The shielded container (1) according to claim 1, wherein it comprises a plate (8) added against the outer face of the container body bottom element (21), said plate (8) comprising an anti-tilt extension (81) that projects from the plane of the container body rear wall (23), arranged to prevent said shielded container (1) from tipping over at the end of the container lid (3) opening stroke.

10. The shielded container (1) according to claim 1, wherein it comprises a removable, liquid-tight packaging support (12) arranged in said packaging volume (V), in rest on the container body bottom element (21), said packaging support (12) being adapted to receive and wedge a vessel containing a radioactive product.
